(19) United States
(12) Patent Application Publication
(10) Pub. No.: US 2024/0197295 A1
(43) Pub. Date: Jun. 20, 2024

(54) SYSTEM AND METHOD FOR IMMUNE AND MOLECULAR BIOSENSING VIA TOOTHBRUSH

(71) Applicant: Immune Labs LLC, Encinitas, CA (US)

(72) Inventors: Marcelo Freire, Los Angeles, CA (US); Adir Abergel, Los Angeles, CA (US)

(21) Appl. No.: 18/557,355

(22) PCT Filed: Apr. 28, 2022
(86) PCT No.: PCT/US2022/026699

§ 371 Date: Oct. 26, 2023

(51) Int. Cl. A61B 10/00; A61C 17/00; G01N 33/53

(52) U.S. Cl. CPC A61B 10/0051; A61C 17/00; G01N 33/53; A61B 2010/0006; G01N 2800/18

(57) ABSTRACT

A toothbrush biometric monitoring system includes a multimodal sensing diagnostic system integrally incorporated into an oral hygiene device. The multimodal sensing diagnostic system is configured to capture a target molecule, detect molecular cues, and generate a signal in response to binding of a binding moiety and one or more biomarkers.

### CROSS-REFERENCE TO RELATED APPLICATION

This application claims benefit of priority to U.S. Provisional Application No. 63/180,986, filed Apr. 28, 2021, the disclosure of which is hereby incorporated in its entirety by reference herein.

### TECHNICAL FIELD

The present disclosure relates to a system and method for rapid collection and assay of oral fluids.

### BACKGROUND

The oral cavity is a portal for immunity and infectious diseases, with ample access to external toxins, microbes, metabolites, chemicals, nucleic acids, antigens, and viruses. The oral biofluid is a composite of biomarkers available present in whole saliva. As an essential biofluid to the human physiology, oral fluids are responsible for shaping oral and systemic health, including gastrointestinal health, brain, cardiovascular, liver, pregnancy, sexual transmitted diseases and other systemic conditions. Oral pathology has also been linked to acute, chronic and metabolic diseases including type 2 diabetes, cardiovascular disease, cancer, preterm labor and Alzheimer's. In addition, the oral fluid systemic communication to other parts of the body is related to impacting the severity of the disease. The oral cavity is also of major importance for human health and well-being.

### SUMMARY

A toothbrush as biometric monitoring system to sense systemic health and disease. A platform diagnostic system integrally incorporated into an oral hygiene device and configured to capture targeted molecules, an oral fluid collector coupled with an absorbing layer configured to capture analytes of a biological fluid, a device base functioning as multimodal sensing layer configured to detect molecular cues, a recognition moiety configured to bind one or more analytes, wherein the binding moiety interacts with one or more biomarkers present in the biological fluid, an electrically conductive surface electrically coupled to the recognition moiety and configured to generate a signal in response to binding of the binding moiety and the one or more biomarkers, an amplifying equipment to detect and amplify nucleotide molecules, wherein the signal is indicative of a parameter value of each of the one or more biomarkers, and a logic circuit communicatively coupled to the electrically conductive surface and a display, wherein the logic circuit is configured to receive the signal from the electrically conductive surface and display an indication on the display based on the signal.

A device includes an electrically conductive layer having attached thereto one or more binding moieties configured to bind one or more biomarkers of a biological oral fluid, wherein binding of the one or more biomarkers to the binding moieties generates a change in one or more electrical characteristics of the conductive layer, one or more base layers disposed in contact with the electrically conductive layer, wherein the one or more base layers comprise at least one of ceramic, silica, polymer, or plastic, and a logic circuit coupled to an antenna, the logic circuit coupled to the electrically conductive layer, wherein the logic circuit is configured to, in response to the change in one or more electronic characteristics of the electrically conductive layer caused by the binding of the one or more biomarkers, transmit a signal, via the antenna or airdrop system, to an external device, and wherein the signal is indicative of the change.

A device includes a housing including an electrically conductive layer having attached thereto one or more binding moieties, wherein the one or more binding moieties are configured to bind with one or more biomarkers, wherein binding of the one or more biomarkers to the one or more binding moieties generates a change in at least one electrical characteristic of the electrically conductive layer, and wherein a communication circuit disposed within the housing is configured to, in response the change in the at least one electrical characteristic caused by the binding of the one or more biomarkers to the one or more binding moieties, transmit a signal to an external device based on the change.

### BRIEF DESCRIPTION OF THE DRAWINGS

The detailed description particularly refers to the following figures, in which:

FIG. 1 is a diagram illustrating an example implementation of a toothbrush diagnostic system of the present disclosure;

FIG. 2 is a diagram illustrating a perspective view of the system of FIG. 1;

FIG. 3 is a diagram illustrating an exploded view of the system of FIG. 1; and

FIGS. 4-8 are diagrams illustrating example implementations of the system of FIG. 1.

### DETAILED DESCRIPTION

Not only does the oral cavity provide entry to the digestive and respiratory systems, but it also provides a portal for immunity, and a protective barrier from invading pathogens. More than 2000 oral bacterial, archaeal, fungal and viral species have been identified to-date in oral fluids, with evidence of 296 species-level taxa in a typical individual's mouth, and these are referred to collectively as the human oral microbiome. The species are transient or colonize specific niches, including tongue, oral mucosa, mineralized tooth surfaces, sublingual, vestibular and periodontal tissues. While some microbial community members are considered pathobionts which directly cause diseases to specific niches, including dental caries (tooth decay), periodontal diseases (gingival inflammation), and oral pathologies (oral inflammation and cancers), most microbes behave opportunistic when in contact to oral fluids, prior to developing niche tropism.

There is a need for home adapted devices to monitor complex exposures to infectious agents and improved understanding of the immune and molecular interactions governing health and disease transitions. Especially, home laboratory devices that bring science to the everyday use. Conventional oral devices, such as toothbrushes, are typically manual or electrical with vibration or specialized functions that are focused on oral hygiene. A single main switch for the operating motor may be pushed or automatically improve the user operation to advantageously require fewer inputs to the user. The system of the present disclosure includes a diagnostic system within an oral hygiene device and offers a user monitoring of biomarkers molecular information (e.g., diagnosis and monitoring of patient infection to SARS-Cov-2, Influenza, Streptococcus).

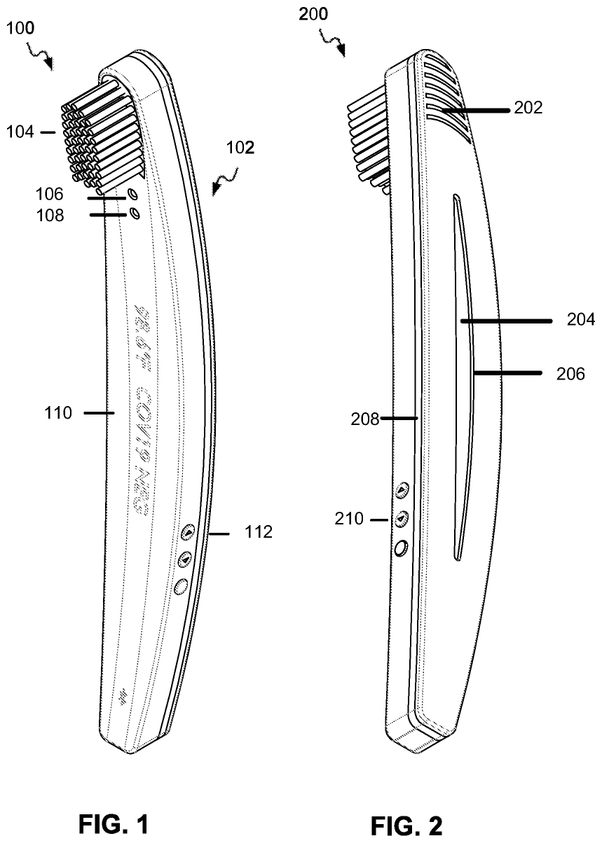

FIG. 1 illustrates an example implementation of a biometric oral monitoring and diagnostic system 100 of the present disclosure. The system 100 includes a housing 102. In some instances, the housing 102 may comprise an oral hygiene device, such as, but not limited to, a toothbrush. The system 100 includes a multimodal sensing diagnostic system including an oral fluid collector and a sensing device. In other examples, the housing 102 of the system 100 and/or the multimodal sensing diagnostic system may include a replaceable bristle head 104, a camera 106, an IR camera 108, a display 110, and a plurality of user controls 112.

FIG. 2 illustrates a perspective view 200 of the multimodal sensing diagnostic system and the system 100 in accordance with the present disclosure. The toothbrush, oral fluid collector, and sensing device attached to the housing 102 include a saliva collector 202, a clear results window 204, a test result 206, a plastic seal 208, and a plurality of user controls 210.

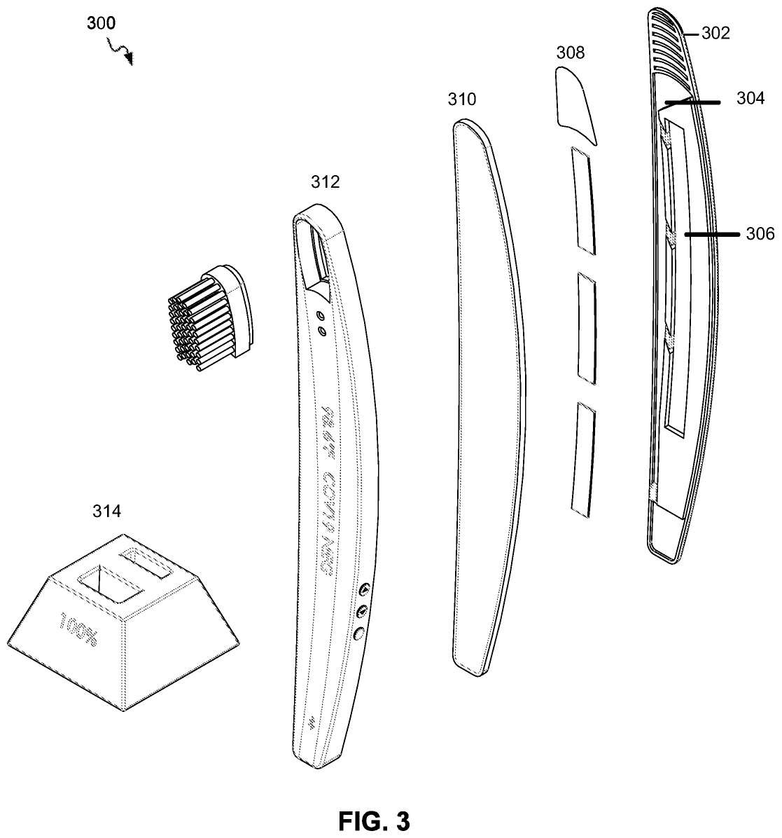

FIG. 3 illustrates an exploded view 300 of the system 100 in accordance with the present disclosure. The multimodal sensing diagnostic system and the system 100 includes an oral fluid collector, a filtering system, a plurality of detecting strips 308, a transparent cover seal 310, and a toothbrush device 312. In an example, the multimodal sensing diagnostic system and the system 100 may include a replaceable bristle attachment 316, a docking station 314, a saliva collector 302, a saliva channel 306, a clear window 304, a filter, and detecting test strips 308, a transparent cover seal 310, a toothbrush device, a charging docking device with a hidden LED display.

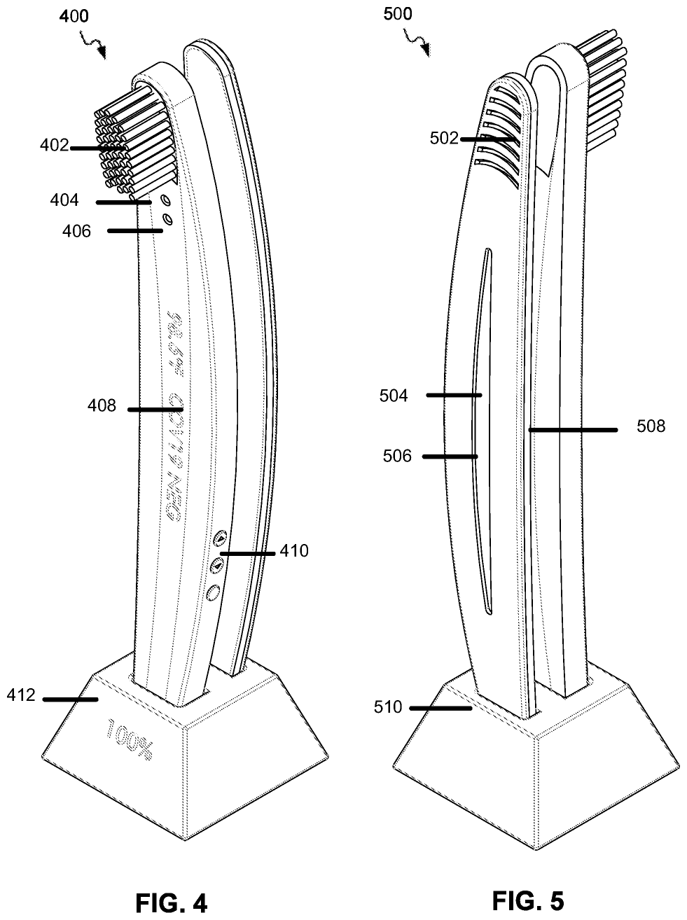

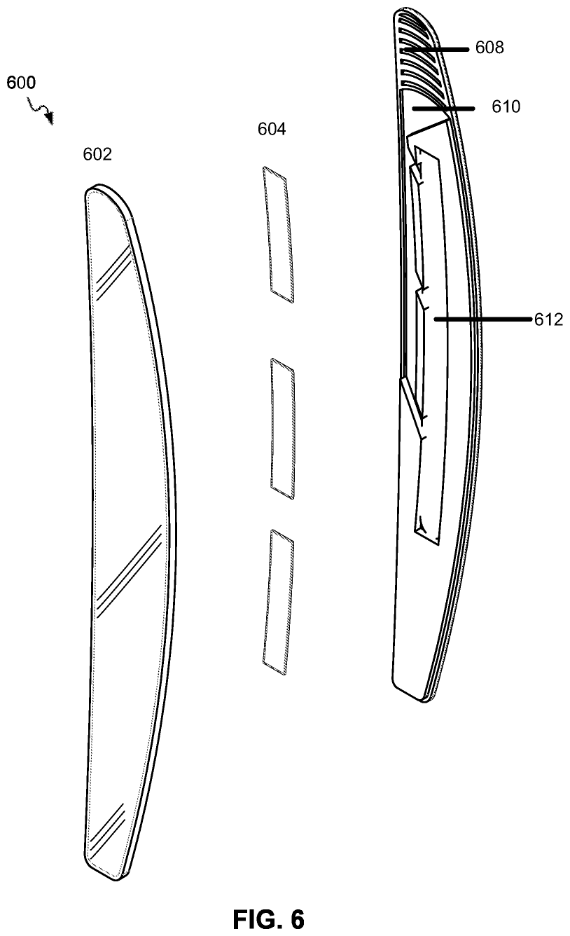

FIGS. 4 and 5 illustrate views 400 and 500, respectively, of the multimodal sensing diagnostic system and the system 100 in accordance with the present disclosure. The system 100 may include a charging station 402 and a testing device 404. The housing 102, along with the oral fluid collector and the sensing device, may be selectively removed from the charging station 402. A replaceable bristle head attachment 403, a camera 405, an IR camera 406, an LED display 408, and a plurality of user controls 410. The charging docking cradle 412 may include an LED display 414. As illustrated in FIG. 5, the system 100 of the present disclosure includes a saliva collector 502, a plurality of clear windows 504, a test result display 506, a plastic seal 508, and a PCR docking station 510.

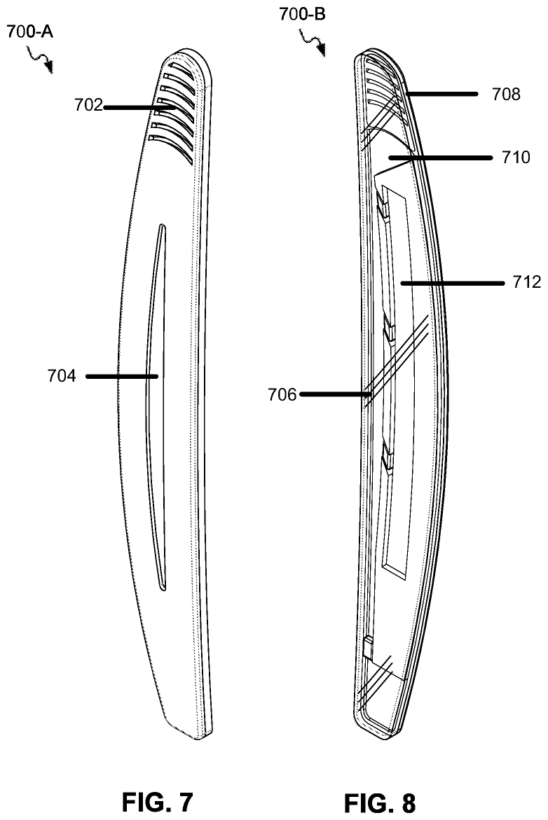

FIGS. 6-8 illustrate example implementations 600 and 700 of the multimodal sensing diagnostic system and the system 100 in accordance with the present disclosure, respectively. The implementation 600 includes a transparent cover 602, a plurality of test strips 604, a saliva collector 606, a saliva channel 608, and a clear window 610. The implementations 700-A and 700-B include a saliva collector 702, a clear view 704, a plastic seal 706, a saliva channel 710, and a clear window 712.

In addition, in some embodiments, the present disclosure provides a method of sensing the temperature of the subject's body, wherein infrared sensors may be configured to capture temperature information from saliva, oral mucosa, skin, but still maintaining the shape of regular toothbrushes. At least one sensor is attached to the toothbrush and the signal is transmitted to the user's device.

Resistance temperature detectors (RTD) are highly accurate temperature sensors. These quick sensing elements are able to utilize the relationship between resistance and temperature references. Micro-RTD elements embedded in a handle and/or in a head of a toothbrush provides long-term stability, waterproof properties with availability in different dimensions. Thus, temperature monitoring from the mouth and skin (e.g., forehead or hands) may monitor the body's temperature and clinically relevant changes, including fevers.

An aspect of the disclosure relates to a method of fabricating toothbrushes and oral devices, including the addition of analyte sensing systems. The method comprises an oral fluid sensor array to facilitate the monitoring of the body's health and diagnose diseases. The method comprises attaching an absorbing pad, filtering system, buffering, and sensing layers to a toothbrush and oral device, and facilitating the diagnostic and feedback. The method includes dissecting, separating, or otherwise discerning biological markers from oral fluid analytes, where the oral fluid includes one of salivary, gingival, lymphatic, spinal, interstitial fluids or sweat.

Molecular probes with defining areas of immune conjugation and molecular probes of nucleotide and protein hybridization may either be a direct signal to a sensor or may be embedded to an amplified response to be detected. To capture response from this conjugation, and/or hybridization, this change in color, texture, shape, volume is captured by specific sensing device attached as a dock system. The base of this device may serve as charging station and also with transmitting and displaying system, providing the results to the user via smart phone and computer devices.

In some embodiments, the multimodal sensing layer comprises an immune conjugate, for example, antibody-antigen immune complex, peptide-peptide conjugation. In some embodiments, the probe is derived from nucleic acid for RNA and DNA amplification. In some embodiments, protein and peptide derived conjugations may be tagged with specific enzymes that provide catalytic regions with colorimetric change, for example luciferase. In some embodiments, the multimodal sensing layer may be regenerated in-situ, for example by renewed enzymatic activity.

A system for detecting a target molecule in a biological sample includes an attachment as part of the device having lateral flow immunoassay that may comprise multiple lanes extending along a length of the device. In some embodiments, a system for detecting a target molecule in a biological sample may include temperature derived amplification, for example, PCR (polymerase chain reaction). In some embodiments, a method for detecting a target molecule in a biological sample may include microfluidics layer, allowing the fluids and hybridizations to occur prior to molecular tags recognition. With the logic circuit, an indication that the target molecule is present in the biological sample-based measurement of the biochemical property.

Such devices may include physiological sensors configured to sense certain physiological parameters of the use, such as heart rate, blood pressure, via infrared monitoring and anatomy of the oral cavity organs via a miniaturized camera. An interface may be configured to transmit information corresponding to the conditioned sensor signals to remote computing devices using, for example, Bluetooth™, ultrasound, global positioning system (GPS), and other wired or wireless formats of transmission.

The biometric monitoring and diagnostic device include a diagnostic attachment to a toothbrush, a plurality of oral fluid analyte, substrates designed to allow fluidics, buffering, and detection sensors configured to sense a plurality of different analytes of a subject at a substantially the same time.

For example, in some embodiments, the biometric monitoring and diagnostic device comprises a replaceable toothbrush-bristle head and a main body to which the bristle head is attachable, substantially as illustrated in the exemplary drawings submitted herewith. The main body is sized and shaped for comfortable grasping by a user while the user brushes his or her teeth. The bristle head may include a locking feature, such as a lip, configured to interact with a corresponding structure on the main body to ensure secure attachment of the bristle head to the main body. In some embodiments, the front and/or rear surfaces of the main body each may comprise a strip of soft-touch material to aid grasping by the user and/or overlie a hidden reader housed within the main body. One or more control buttons (e.g., “up,” “down,” “select”) may be defined on a side surface of the main body in operative connection with control circuitry housed within the main body and enabling the user to select a desired operation of the oral device.

In some embodiments, a rear surface of the main body adjacent the bristle head defines a plurality of openings that comprise of an oral fluid collector. During use, a sample of the user's oral fluid (e.g., saliva) enters the saliva collector. In some embodiments, a filtering system is disposed adjacent/within the saliva collector to filter out cells and macro particles from the biological sample before it passes into a saliva channel defined within the interior of the main body and contacts the multimodal sensing layer contained within the main body. Results of the sensing operations then may be displayed via a clear window embedded within a rear surface of the main body and/or an LED display positioned at a front surface of the main body. The display may be configured to be visible through the material of the front surface of the main body while in use and be substantially invisible while not in use.

In some embodiments, the miniaturized camera (e.g., visible-light camera or infrared (IR) camera) may be housed within the main body with lens(es) thereof positioned at the front surface of the main body to face the user during use. In any such embodiments, the biometric monitoring and diagnostic device may be powered by any suitable power source, such as a rechargeable battery housed within the main body. In such embodiments, the main body may be configured for reception within a charging station configured to supply current to the rechargeable battery. The combinatorial role of surveying body's temperature with image capturing of the subjects' tissues, including, but limited to, oral mucosal blood vessels have also not been developed. Oral fluid collection combined with fluidics technology presents mechanical, electrical, immune and molecular sensing properties in wet environments. Democratization of diagnostics by adding home devices can facilitate human health monitoring, and may improve molecular understanding of treatments, and help predict population pandemic.

The present disclosure relates to a system and method for rapid collection and assay of oral fluids to improve home diagnostics and monitoring of medical and dental health. As a personal diagnostic device employed to detect immune, microbial, molecular, physiological, and pathological cues to personalized and precision medicine and dentistry. The non-invasive monitoring oral device provides the individual with both cross-sectional and longitudinal personalized data from biomarkers, temperature, and camera insights important to health and disease. A signal conditioner is coupled to the uniplex sensor and multiplex sensor arrays attached to the oral device. Through an interface configured to transmit information from the conditioned sensors, information regarding infectious diseases, immune responses, metabolic changes, viruses, microbial dysbiosis, and genetic conditions may be transmitted to the phone and computer device. This invention also relates to systems providing feedback to the subject while capturing oral biofluids to test the immune, microbial, and molecular content, ultimately providing better understanding of health.

Molecular systems to detect oral fluid changes have not been incorporated in mainstream diagnostics, and not added to home laboratory strategies, or devices for personal use. There is an eminent need to understand how microbes and infectious agents behave in the mucosal tissue. Since most microbes have evolved with host human host tissues over thousands of years and are specifically adapted to the mucosal surfaces, the human oral cavity is a favorable home to one of the most complex microbial ecosystems within the body. Recent estimates suggest there are more than 800 bacterial species, millions of viruses, and yeast in the normal human flora of the oral cavity, many of which have been implicated in local and systemic diseases. The sequential organization of the oral microbiome is complex, niche-dependent, and distinct in health and disease states. For example, the tooth surface may be coated with salivary mucins and proline-rich proteins. While the tongue is colonized by specific biofilm species. Other proteins such as mucins act to aggregate and clear the bacteria from oral surfaces. These concepts are complex and can be monitored or detected in an individualized fashion.

Although biomechanical and biochemical cues control initial adhesion of microbial biofilms on hard and soft tissue surfaces, multiple signaling molecules, such as cytokines, facilitate and/or protect microbial colonization as well as participate in the development and maintenance of immunity at the mucosal border. Thus, oral fluids contain information from microorganisms and beyond microbes including cells from the immune system. For example, the gingival crevice has been shown to express abundance of interleukin (IL)-8 cytokines after releases from the junctional epithelium. This gradient helps recruit immune cells, for example neutrophils, into the tooth gingival pocket where bacteria naturally accumulate. Once in the gingival sulcus/pocket, immune cells form a barrier between the junctional epithelium and the subgingival biofilm. At this point the content released from the immune cells, impact the content of saliva. Functionally, these cytokines impact cellular fat. In the case of IL-8, it prevents the migration of the bacteria deeper into soft tissues and prevent reaching to major blood vessels, and this process is essential to maintain oral-systemic homeostasis. Until this day, traditional methods evaluate oral fluids ex vivo are not able to capture the complexity and dynamics of oral fluids on-site and on a real-time and on a personalized basis.

The composition of the oral fluid includes saliva, crevicular fluids, blood, serum, interstitial fluids, host cell secretions, microbial contents, water, and oral sweat. This rich content provides unique diagnostic value to the subject and the population. Saliva provides an optimal buffering system for microbial acidic metabolites, maintaining microbial community oral microbiota homeostasis in a neutral pH and a constant supply of nutrients to the environment. However, microorganisms are sensitive to even minor changes in the local environment, including pH, nutrients, oxygen, moisture, and host immune responses, for instance, changes in saliva flow rate (e.g., in dry mouth patient) or pH (e.g., during soft drink consumption) may dysregulate its buffering capacity, and prolonged changes in the oral microenvironment over time may result in shifts in the microbial composition, and some pathogenic bacteria may become dominant in their local community. Additionally, many salivary components are known to affect bacterial growth, e.g., pH modulating molecules such as bicarbonates, phosphates, and urea, that modulate the pH in the oral cavity; and many salivary proteins contribute to processes associated with overall oral microbial metabolism, aggregation, attachment and clearing. Saliva as part of the oral fluid also provides necessary moisture and is slightly acidic to neutral pH (6-7) for pathogen growth and even microorganism development. Monitoring of saliva has implications to understanding in more detail how these changes within an individual occur in a personalized manner.

Due to continuous production by major and minor salivary glands, with approximately release 1 and ½ liters produced daily, the oral fluid provides a source of biological material for identification of infectious diseases, metabolic changes, inflammatory and autoimmune events, genetic and rare conditions, and sexually transmitted diseases. In addition, it is accepted as a non-invasive source for daily and continuous longitudinal monitoring. In contrast with blood, collection of saliva is minimally invasive. Levels of certain biomarkers may be elevated or may otherwise vary as compared to a predefined biomarker value that occurs normally or abnormally, prior to manifestation of a disease. Based on a comparison between a given predictive biomarker value and an actual sensed biomarker value, biomarkers from oral fluids may be used to predict disease risk, disease initiation, improving precision in diagnostics, prognostics, and adopted treatment plan. Examples of biomarkers include, but are not limited to, a biochemical or molecular measurement of a normal physiological and pathogenic process (e.g., proteins from influenza virus, SARS-CoV-2, and streptococcus infection). Examples of biomarkers also include forensic parameters, family tree parameters or identifiers, and a response to exposure to certain conditions, treatments, and/or interventions.

Saliva carries a wide variety of biomolecules, cells, microbes and proteins from diverse origins. Protein analysis is a powerful tool revealing the content of saliva. With a combination of multidimensional technical approaches that are innovative in fractionation (e.g., on either protein or peptide level), mass spectrometric identification (e.g., ionization, fragmentation), and bioinformatics interpretation (e.g., algorithms that convert mass spectrum to amino acid sequence), markers with functional meaning can be detected. A plurality of proteins may be identified from saliva using, for example, in-solution digestion, a 2-hour liquid chromatography (LC) gradient, and a mass spectrometer (MS). As another example, polymerase chain reaction (PCR) assays, in-StageTip digestion, 100-min LC gradient and an MS instrument may be employed to study the saliva proteome content. A single LC-MS run has previously revealed that about 4,000 proteins are present in saliva and eight-fraction fractionation steps revealed that 5,563 proteins are present in saliva. This underscores the complexity of the vast molecular content of saliva. In addition, minor technical improvements such as ultra-long LC column separation (e.g., LC column separation greater than 40 cm), double digestion, and tip-based sample processing with minimal sample loss have also shown significant contributions to the enhanced proteomics analyses. An oral device attached to a diagnostic sensing system of the present disclosure may be configured to perform diagnostics based on new advancements of both nucleotide molecular sensing and proteomic assays for the purposes of health monitoring, and diagnostics.

Conventionally, saliva tests have been accepted by the public and most recently by the medical field for swabbing and sampling the liquid in/on cup/containers to assay infectious diseases, pregnancy tests, drug testing, genomics and others. Saliva and posterior oropharyngeal swab sampling methods are less invasive and most acceptable to patients. The non-invasive component is attractive for host testing and longitudinal capability to develop a curve of their own data. This approach also reduces risks and exposure for healthcare workers as compared to nasopharyngeal swab sampling. Traditional SARS-CoV-2 testing on saliva samples collected at home using a designated self-collection kit may require shipment to a laboratory. The feasibility and reliability of point of care testing depends on the simplicity and accuracy of the assay, training of the personnel to carry out the experiment, quality and availability of instruments, and integration of testing to shipment and results.

The incorporation of home device for diagnostics for immune and molecular diagnostics, leveraging the biofluid monitoring into an everyday toothbrush has not been developed. Further, employing a reliable point-of-care platform can facility direct diagnostic testing for individual purposes. The embodiment is not limited to home testing. Since toothbrush is a device used during travel, commercial environments, this innovation has potential impact to populational health monitoring while the subject is travelling and working. For example, data of from multiple devices could impact comparisons from a nationhood, or different states, countries Data from the testing devices could lead to modeling of travelling patterns and detection emerging infectious diseases globally.

While the diagnostic concepts of the present disclosure are susceptible to various modifications and alternative toothbrush forms, specific exemplary embodiments are shown by way of example of a diagnostic toothbrush sensing device in the drawings and are described. It should be understood, however, that there is no intent to limit the concepts of the present disclosure to the particular forms disclosed; on the contrary, the intention is to cover all modifications, equivalents, and alternatives falling within the spirit and scope of the invention as defined by the appended claims.

References in the specification to “one embodiment,” “an embodiment,” “an illustrative embodiment,” etc., indicate that the described embodiment may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one A, B, and C” can mean (A); (B); (C): (A and B); (B and C); (A and C); or (A, B, and C). Similarly, items listed in the form of “at least one of A, B, or C” can mean (A); (B); (C): (A and B); (B and C); (A and C); or (A, B, and C).

The disclosed embodiments may be implemented, in some cases, in hardware, firmware, software, or any combination thereof. The disclosed embodiments may also be implemented as instructions carried by or stored on one or more transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

While the disclosure has been illustrated and described in detail in the drawings and foregoing description, such an illustration and description are to be considered as exemplary and not restrictive in character, it being understood that only illustrative embodiments have been shown and described and that all changes and modifications that come within the spirit of the disclosure are desired to be protected.

The method here described advance the conventional daily use of the oral toothbrush into a laboratory platform providing oral detection of body conditions or disorders. By incorporating of an absorbing layer to capture analytes from oral fluid, and the multimodal sensing layer to detect molecular cues, the regular process of oral hygiene is transformed to a monitoring method and system. Because the oral fluids content includes antigens, antibodies, nucleic acids, lipids and overall biomarkers, the biometric oral monitoring can process the data, while a calculation of the individual levels is further compared to its baseline, in addition to comparisons to populational levels. This score system may simplify the user interpretation of their own values (e.g., low, moderate, high), ultimately facilitating better interpretation of the subject's health via individualized monitoring. The method here described applies for cross-sectional monitoring (e.g., positive versus negative), and longitudinal monitoring (e.g., “viral load is lower than last week”, or “antibody levels are higher than yesterday”). This has ultimate impact to one's health, environmental, medication and dietary changes, thus improving how medicine interpretates individualized monitoring system.

There is a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A biometric oral monitoring system comprising:
an oral hygiene device including a display;
a multimodal sensing diagnostic system integrally incorporated into the oral hygiene device and configured to capture and evaluate at least one biomarker relative to normal and abnormal levels in a human, the multimodal sensing diagnostic system including:
an absorbing layer configured to capture analytes of a biological oral fluid,
a multimodal sensing layer configured to detect molecular cues,
a recognition moiety configured to bind to one or more analytes, wherein the binding moiety interacts with at least one biomarker present in the biological fluid to detect the at least one biomarker,
an electrically conductive surface electrically coupled to the recognition moiety and configured to generate a signal in response to binding of the binding moiety and the at least one biomarker, wherein the signal is indicative of a parameter value of the at least one biomarker, and
a logic circuit communicatively coupled to the electrically conductive surface and the display, wherein the logic circuit is configured to receive the signal from the electrically conductive surface and display an indication on the display based on the signal.

2. The biometric oral monitoring system of claim 1, wherein the multimodal sensing layer is one of antigenic, immunological, infectious agent-, and molecular-based.

3. The biometric oral monitoring system of claim 1, wherein the biological oral fluid is one of saliva, crevicular fluid, lymphatic fluid, blood, interstitial fluid, spinal fluid, dental plaque, and oral sweat.

4. The biometric oral monitoring system of claim 1, wherein the at least one biomarker comprises at least one of a host immune molecule, an immunoglobulin, a virus, a nonviral pathogen, a bacterial metabolite, a bacterial membrane component, a bacterial cell wall component, a polysaccharide, and a nucleic acid.

5. The biometric oral monitoring system of claim 1, wherein the electrically conductive surface detects binding of the binding moiety and the at least one biomarker by detecting a change in at least one of an electrical current, an electrical voltage, and an electrical impedance.

6. The biometric oral monitoring system of claim 1, wherein the oral hygiene device comprises one of plastic, polymer, titanium, and ceramic material.

7. The biometric oral monitoring system of claim 6, wherein the oral hygiene device is shaped for use in an oral cavity.

8. The biometric oral monitoring system of claim 7, wherein the oral hygiene device is shaped for use in one of buccal regions, a sublingual region, a palatal region, and a vestibular region of the oral cavity.

9. The biometric oral monitoring system of claim 1, wherein the logic circuit is configured to transmit a signal indicating a presence and/or concentration of the at least one biomarker to an external device.

10. The biometric oral monitoring system of claim 6, wherein the biological oral fluid originates in tissue of the oral cavity.

11. A device comprising:
an electrically conductive layer having attached thereto one or more binding moieties configured to bind one or more biomarkers of a biological oral fluid, wherein binding of the one or more biomarkers to the binding moieties causes a change in one or more electrical characteristics of the conductive layer;
one or more base layers positioned in contact with the electrically conductive layer, wherein the one or more base layers comprise at least one of ceramic, silica, polymer, or plastic; and
a logic circuit coupled to an antenna, the logic circuit coupled to the electrically conductive layer, wherein the logic circuit is configured to, in response to the change in one or more electrical characteristics of the electrically conductive layer caused by the binding of the one or more biomarkers, transmit a signal, via the antenna, to an external device, and wherein the signal is indicative of the change.

12. The device of claim 11, wherein the biological oral fluid is at least one of saliva, crevicular fluid, lymphatic fluid, blood, interstitial fluid, spinal fluid, dental plaque, and oral sweat.

13. The device of claim 11, wherein the one or more biomarkers comprise one or more inflammatory molecules, a viral metabolite, a bacterial membrane component, a bacterial cell wall component, a virus, a nonviral pathogen, a polysaccharide, a nucleic acid, or any combination thereof.

14. The device of claim 11, wherein the signal indicates a presence and/or concentration of the one or more biomarkers.

15. The device of claim 11, further comprising a bristle attachment detachably coupled to one of the electrically conductive layers and the one or more base layers.

16. The device of claim 11, wherein the external device is configured to receive and analyze the signal indicative of the change in the one or more electrical characteristics of the conductive layer.

17. The device of claim 16, wherein analyzing the signal includes identifying a normal or an abnormal medical condition.

18. A method for detecting a condition or disorders associated with oral and systemic health in a subject, the method comprising:
detecting altered level of at least one biomarker related to monitoring of homeostasis of diagnostic infectious diseases and immunological changes by detecting binding of the one or more biomarkers to one or more binding moieties that causes a change in at least one electrical characteristic of an electrically conductive layer, and
in response the change in the at least one electrical characteristic caused by the binding of the one or more biomarkers to the one or more binding moieties, transmitting a signal indicating the change.

19. The method of claim 18 further comprising
applying oral fluids or orally derived molecules to a device;
capturing the oral fluids or the orally derived molecules;
processing a cellular and molecular content of the oral fluids or the orally derived molecules to detect fixation, hybridization, and stabilization;
conjugating one or more binding moieties by detecting one of the antibodies, a binding fragment of the antibody, and a nucleic acid, wherein detecting the binding includes detecting tags or labels, and wherein detecting labels is using an optical sensor.

20. The method of claim 18, wherein the housing includes attachment to a diagnostic that is coupled with wither an analog layer or electrically conductive layer to isolate and detect nucleic acid, proteins, and metabolites from oral fluids, further comprising at least one of
detecting the level of one or more biomarkers from oral fluids to infer immune health or dysbiosis,
calculating the levels of subject to provide individualized data in a cross-sectional basis, comparing to previous testing bassline to provide a data curve a longitudinal basis,
providing a score system of health, risk for disease, and disease to facilitate better compression of complex molecular data, and
providing information on biomarkers to detect the condition or disorder in the subject.
